(19) United States
(12) Patent Application Publication
(10) Pub. No.: US 2018/0296625 A1
(43) Pub. Date: Oct. 18, 2018

(54) PHARMACEUTICAL COMPOSITION TREATING SNORING, PREPARATION METHOD THEREFOR AND USE THEREOF

(71) Applicant: SICHUAN SUN & HEALTH BIOTECH CO., LTD, SICHUAN (CN)

(72) Inventors: XIAOXUE GUO, Sichuan (CN); XIAOLING YAN, Sichuan (CN); LISHA ZHONG, Sichuan (CN); HONG ZHANG, Sichuan (CN); YUCHAN ZHONG, Sichuan (CN); ZEXIANG HUANG, Sichuan (CN); MINGXIANG TAN, Sichuan (CN)

(21) Appl. No.: 15/527,612

(22) PCT Filed: Sep. 10, 2015
(86) PCT No.: PCT/CN2015/089333

§ 371 Date: May 17, 2017

(30) Foreign Application Priority Data
Sep. 16, 2014 (CN) 201410473010.7

(51) Int. Cl. A61K 36/74; A61P 11/00; A61K 36/482; A61K 36/428; A61K 36/899; A61K 36/9066; A61K 9/00; A61K 9/16

(52) U.S. Cl. CPC A61K 36/74; A61P 11/00; A61K 36/482; A61K 36/428; A61K 36/899; A61K 2236/39; A61K 9/0053; A61K 9/16; A61K 2236/33; A61K 2236/331; A61K 36/9066

(57) ABSTRACT

The present invention relates to a pharmaceutical composition for snoring treatment which comprising 15-25 doses of Gambir and 5-15 doses of Cassia. Fabrication of the composition is also described. The present invention relates to Gambir and Cassia and their compositions with Trichosanthes, Turmeric and Phragmites, which has remarkable effects on improving or inhibiting snoring and has no side-effects. It can be a new choice for clinical treatment of snoring since it has no surgery risks.

This application is submitted to State Intellectual Property Office of the People's Republic of China on Sep. 16 2014. Application No. is 201410473010.7. Privilege of application of patent for title of invention of “A pharmaceutical composition for snoring treatment, its fabrication and utilities”, the whole content of which is quoted and stated in this application

### FIELD OF THE INVENTION

The present invention relates to a pharmaceutical composition for snoring treatment, its fabrication and utilities

### BACKGROUND

Snoring (obstructive sleep apnea in medical science) is a ubiquitous sleeping phenomenon. At present, most people take it as a common behavior and even think it is a sign of good sleep.

Reports show that at least 20% people snore during sleeping. In fact, snoring is harmful to heath, because snoring causes repetitive sleep apnea which is associated with reduction in blood and brain oxygen saturation, thus causing hypoxaemia and possibly hypertension, brain diseases, arrhythmia, myocardial infarction and stenocardia. Sudden death could happen if breathing during sleeping in the night pauses for over 120 seconds.

Medical research shows that snoring is mainly caused by the following three reasons:

1. Central diseases

2. Obstructive diseases

3. Combination of the above two called mixed

Generally speaking, adults' snoring is caused by mixed reasons while children's is caused by obstructive diseases. In-depth studies have determined the risk factors such as smoking, alcohol, obesity and other physiological causes (like deflection of nasal septum, other abnormalities in internal nasal structure, chronic nasal congestion, congestion in laryngeal tissue, fitness, lack of muscle tone, hypertrophy of tonsil and adenoid, and tongue swelling). Drug treatment (benzodazines and sedatives, for instance) can potentially cause muscular flaccidity, thus increase risk or tendency of snoring. Snoring is often categorized into several degrees ranging from mild to severe, including hypopnea (breathing could become astonishingly slow and shallow during this time) and apnea (breathing stops or pauses during this time). The latter is the symptom of obstructive sleep apnea (OSA) which relates to complete or partial obstructions of upper airway.

Currently, treatments for snoring are mainly surgery, drug therapy, physical intervention, etc. Surgery treatments: varied and must be chosen according to the specific airway obstructions. Nasal surgery and palatopharyngoplasty are the most common surgeries taken to treat snoring and sleep apnea. However, whether it works or not depends on the patient. This surgery also makes patients suffer a lot. Drug treatment: there are plenty of drugs domestic and overseas used to treat snoring, such as acetazolamide and thyrine. By boosting metabolism, these drugs have certain effects alleviating snoring to some extent during the therapy, but fail to cure. Physical instrument like nasal splint and cingulum rest appeared in recent years is seen less and less on market due to its limited effectiveness.

Chinese traditional medicines have years of history in treating snoring, with remarkable effects, no side-effect and extensive application.

### CONTENTS OF THE INVENTION

The present invention is aimed to provide a pharmaceutical composition for snoring treatment, its fabrication and utilities.

Specifically, the present invention provides a pharmaceutical composition for snoring treatment. The crude drugs of it contain the components proportioned by the weight of:

15-25 doses of Gambir and 5-15 doses of Cassia.

The present invention unexpectedly found during study that using Gambir together with Cassia has better effect than using just Gambir, which shows that the two drugs have synergistic effect when used together in the present invention. What is more, there's no research that reports the advantages of Cassia and cassia twig, being two different parts of a plant, for snoring treatment. The present invention found out that using Cassia with Gambir generates better effects than using cassia twig with Gambir.

Gambir is dry stem and branch with hook of Uncaria rhynchophylla (Miq.) ex Havil, sweet and a bit bitter in taste and mild cold-natured. It is useful for clearing heat, calming liver wind and relieving convulsion. Modern pharmacology research has proved that Gambir can treat asthma, children convulsion, heating, and dizziness and regulate central nervous system.

Cassia is dry bark of Cinnamomum cassia Presl, acrid in taste and warm-natured. It is used to supplement kidney-yang, warm the stomach and spleen, remove internal cold and promote blood circulation. Modern pharmacology research has proved that Cassia can treat asthma and regulate central nervous system.

Crude drugs of the composition in the present invention contain components proportioned by the weight of:

15 doses of Gambir and 5 doses of Cassia; 20 doses of Gambir and 10 doses of Cassia; 25 doses of Gambir and 15 doses of Cassia.

In a detailed fabrication for the present invention, weights of of Gambir and Cassia are: 25 doses of Gambir and 15 doses of Cassia.

The present invention further found that, adding Trichosanthes, Phragmites and Turmeric to Gambir and Cassia increases effect on treating snoring, compared with using same amount of just Gambir and Cassia. Therefore, crude drugs of the composition in the present invention may contain components proportioned by the weight of: 12-18 doses of Trichosanthes, 12-20 doses of Phragmites and 6-12 doses of Turmeric.

Among which:

Trichosanthes is the root of Trichosanthes kirilowii Maxim, sweet and a bit bitter in taste and mild cold-natured. It is used to treat pyreticosis, polydipsia, lung heat, dry cough, internal heat, symptom-complex of excessive eating, swelling and ulcer on the body surface and pyogenic infections. Modern pharmacology research has proved that Trichosanthes is effective at anti-early pregnancy, anticancer, improving immunity, and antibiosis and antivirus.

Phragmites is fresh or dry rhizome of Phragmites communis Trin, sweet in taste and cold-natured. It is used to treat pyreticosis, polydipsia, stomach heat, emesis, lung heat, dry cough, pulmonary abscess, heat strangury and pain in gonorrhea. Modern pharmacology research has proved that Phragmites is effective at restraining skeletal muscle and relaxing isolated guinea pig ileum.

Turmeric is the dry rhizome of Curcuma Longa L., acrid and bitter in taste and warm-natured. It is used to treat chest pain, abdominal pain, and dysmenorrhea and limb pain caused by Qi-stagnation and blood stasis. Modern pharmacology research has proved that Turmeric has hypolipidemic, antineoplastic, anti-inflammatory and antiviral effects. It also has cholagogue effects and helps promoting cardiovascular system.

Furthermore, crude drugs of the composition in the present invention may contain components proportioned by the weight of:

15 doses of Gambir, 5 doses of Cassia, 12 doses of Trichosanthes, 12 doses of Phragmites and 6 doses of Turmeric;

20 doses of Gambir, 10 doses of Cassia, 15 doses of Trichosanthes, 17 doses of Phragmites and 9 doses of Turmeric;

25 doses of Gambir, 15 doses of Cassia, 18 doses of Trichosanthes, 20 doses of Phragmites and 12 doses of Turmeric;

The composition is fabricated by combing the active ingredients such as power, aqueous extract and alcohol extract of the crude drugs with common pharmaceutical auxiliaries or auxiliary materials.

Pharmaceutical auxiliaries mentioned in the present invention refer to substances in the composition apart from active ingredients, including but not limited to filler (diluent), lubricant (glidant or antitack agent), dispersant, humectant, adhesive, modifier, solubilizer, antioxygen, bacteriostatic agent, emulsifier and disintegrating agent. Adhesives include syrup, Arabic gum, gelatin, sorbitol, tragacanth, cellulose and its derivatives (such as microcrystalline cellulose, sodium carboxymethylcellulose, ethyl cellulose, hydroxypropyl methyl cellulose), gelatin mucilage, syrup, starch slurry and polyvinylpyrrolidone; filler includes lactose, powdered sugar, dextrin, starch and its derivatives, cellulose and its derivatives, inorganic calcium salt (such as calcium sulfate, calcium phosphate, calcium hydrophosphate, calcium carbonate), sorbitol and glycine; lubricants include aerosol, magnesium stearate, talcum powder, aluminium hydroxide, boric acid, hydrogenated vegetable oil and polyethylene glycol; dispersants include starch and its derivatives (such as sodium carboxymethyl starch, sodium glycollate starch, pregelatinized starch, modified starch, hydroxypropyl starches, corn starch, etc.), polyvinylpyrrolidone and microcrystalline cellulose; humectants include lauryl sodium sulfate, water or alcohol; antioxygens include sodium sulfite, sodium hydrogen sulfite, sodium pyrosulfite and dibutyl benzoic acid; bacteriostatic agents include 0.5% phenol, 0.3% cresol and 0.5% chloretone; modifiers include hydrochloric acid, citric acid, potassium (sodium) hydroxide, sodium citrate and buffer agents (including sodium dihydrogen phosphate, disodium hydrogen phosphate, etc.); emulsifiers include polysorbate-80, fatty acid sarbitan, pluoronic F-86, lecithin and fabaceous lecithin; solubilizers include Tween 80, bile and glycerine.

Pharmaceutically auxiliary ingredients mentioned in the present invention, though have certain physiological activity, won't affect the composition or derivative's taking dominant role in treating diseases. They will only have an auxiliary effect which is the result of utilization of the known activity of them. They are in common use for auxiliary treatment in pharmaceutical field. Adding these auxiliary ingredients in use with the composition of the present invention also falls within protection of the present invention.

Furthermore, the said dosage form is the form of gastrointestinal absorption; the dosage form will be medical liquor, oral liquid, drink, granules, powder, pills, troche or capsule.

The present invention also provides fabrication of the said pharmaceutical composition. Here are the steps:

(1) Take crude drugs according to the formula proportion;

(2) Take the crude drugs and mix them up for aqueous extraction, or extract the crude drugs respectively. Then take the aqueous extractives out for use in next step.

(3) Add common pharmaceutical auxiliaries or auxiliary materials into the aqueous extractives and the composition is made.

Furthermore, extraction method used in step (2) is decoction or ultrasound.

Adding either aqueous extractives or powder in drug is a traditional way used in Chinese traditional medicine. After extracted in water, most of the effective components will come out, since water has high dissolving capacity, which will make it easier for the drug to be absorbed in human body and render better effects. Decoction, for instance, is such kind of dosage form. Adding powder, on the other hand, with relatively large surface area, is also good for absorption of effective components inside body. However, since the drug has not been extracted, the effective components has to be dissolved inside the body and then be absorbed, which makes it slower to take effects, compared with aqueous extractives. Meanwhile, it reduces toxic and side effects of the harmful substances in the medicine. It is suitable for long-term therapy. Making the crude powder into pills, for example, is such kind of dosage form. At present, using ethanol as the solvent to extract drugs is the most common way in pharmaceutical process. Ethanol is a semi-polar solvent. Its dissolving capacity falls between that of polar and non-polar solvent. It can dissolve some water-soluble components and some components that can only be dissolved by non-polar solvent. Using ethanol extraction instead of decoction by water can prevent large amounts of inactive substances from coming out, thus increase concentration and extraction efficiency of effective components. But ethanol costs more than water. Therefore, in modern pharmaceutical production, to save cost of production, water decoction is still adopted most. As long as the physiological activity of the aqueous extractives from the composition in the present invention, water decoction, crude powder, ethanol extraction or mix of the former three can be used to fabricate specific dosage form, to meet needs of production and usage.

The present invention also provides the said pharmaceutical composition's utilities in making drugs for snoring treatment.

The present invention provides composition of Gambir, Cassia, Trichosanthes, Phragmites and Turmeric which has remarkable effects on improving or inhibiting snoring and has no side-effects. It can be a new choice for clinical treatment of snoring since it has no surgery risks.

### DETAILED FABRICATION

### Example 1 Fabrication of Pharmaceutical Composition of the Present Invention

Formula: 15 doses of Gambir and 5 doses of Cassia

The present invention provides fabrication of the composition of medicine material and plant extractives mentioned above. The main steps are:

1. Take Gambir and Cassia, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.;

2. Dry the concentrated solution with high-speed centrifuging and spraying drier and make 80-120 meshes of dry extract powder;

3. According to extraction ratio of Gambir and Cassia, mix the dry extract powder of the two evenly at the proportion prescribed in the formula, add appropriate auxiliaries and make them into ordinary troches.

### Example 2 Fabrication of the Pharmaceutical Composition of the Present Invention

Formula: 20 doses of Gambir and 10 doses of Cassia

The present invention provides fabrication of the composition of medicine material and plant extractives mentioned above. The main steps are:

1. Take Gambir and Cassia, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.;

2. Dry the concentrated solution with high-speed centrifuging and spraying drier and make 80-120 meshes of dry extract powder;

3. According to extraction ratio of Gambir and Cassia, mix the dry extract powder of the two evenly at the proportion prescribed in the formula, add appropriate auxiliaries and make them into ordinary capsules.

### Example 3 Fabrication of the Pharmaceutical Composition of the Present Invention

Formula: 25 doses of Gambir and 15 doses of Cassia The present invention provides fabrication of the composition of medicine material and plant extractives mentioned above. The main steps are:

1. Take Gambir and Cassia, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.; 2. Dry the concentrated solution with high-speed centrifuging and spraying drier and make 80-120 meshes of dry extract powder;

3. According to extraction ratio of Gambir and Cassia, mix the dry extract powder of the two evenly at the proportion prescribed in the formula, add appropriate auxiliaries and make them into ordinary granules.

### Example 4

Formula: 15 doses of Gambir, 5 doses of Cassia, 12 doses of Trichosanthes, 12 doses of Phragmites and 6 doses of Turmeric

The present invention provides fabrication of the composition of medicine material and plant extractives mentioned above. The main steps are:

1. Take Gambir, Cassia, Trichosanthes, Phragmites and Turmeric, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.;

2. Dry the concentrated solution with high-speed centrifuging and spraying drier and make 80-120 meshes of dry extract powder;

3. According to extraction ratio of Gambir and Cassia Trichosanthes, Phragmites and Turmeric, mix the dry extract powder of the two evenly at the proportion prescribed in the formula, add appropriate auxiliaries and make them into ordinary troches.

### Example 5

Formula: 20 doses of Gambir, 10 doses of Cassia, 15 doses of Trichosanthes, 17 doses of Phragmites and 9 doses of Turmeric

The present invention provides fabrication of the composition of medicine material and plant extractives mentioned above. The main steps are:

1. Take Gambir, Cassia, Trichosanthes, Phragmites and Turmeric, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.;

2. Dry the concentrated solution with high-speed centrifuging and spraying drier and make 80-120 meshes of dry extract powder;

3. According to extraction ratio of Gambir and Cassia Trichosanthes, Phragmites and Turmeric, mix the dry extract powder of the two evenly at the proportion prescribed in the formula, add appropriate auxiliaries and make them into ordinary capsules.

### Example 6

Formula: 25 doses of Gambir, 15 doses of Cassia, 18 doses of Trichosanthes, 20 doses of Phragmites and 12 doses of Turmeric

The present invention provides fabrication of the composition of medicine material and plant extractives mentioned above. The main steps are:

1. Take Gambir, Cassia, Trichosanthes, Phragmites and Turmeric, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.;

2. Dry the concentrated solution with high-speed centrifuging and spraying drier and make 80-120 meshes of dry extract powder;

3. According to extraction ratio of Gambir and Cassia Trichosanthes, Phragmites and Turmeric, mix the dry extract powder of the two evenly at the proportion prescribed in the formula, add appropriate auxiliaries and make them into ordinary granules.

Experiments are listed below to demonstrate the benefits and effects of the present invention:

Dosages of the test drugs, are calculated by its proportion of corresponding extracted materials in g/kg.

### Experiment 1 Safety and Efficacy Evaluation of Composition of Gambir and Cassia

### I. Safety Evaluation

Products fabricated with composition of the present invention in Example 3 are used in the following experiment.

### 1. Acute Toxicity Test

Give intragastric administration of 50 g test product (crude drug)/kg to lab mouse at one time. The mouse didn't die during the experiment period. Thus it can be concluded that maximum tolerance dose to mouse at one time is 50 g (crude drug)/kg which equals 125 times of clinical daily dosage to human (2 capsules, which contains 12 g of crude drug, one time for man of 60 kg. Daily dosage for man is 24 g which is 0.4 g/kg).

### 2. Long Term Toxicity Test

Give lab mouse intragastric administration of dosage of 20 g/kg for 14 consecutive weeks.

Compared with the control group that are given with distilled water, no clinical symptoms, weight gaining or changes in indicators in hematology and blood biochemical examination, toxicity in organs, system and pathological tissue have been noticed. Four weeks after drug withdrawal, no chronic toxicity has been noticed in the same examination items in the tested animal, which shows that consecutive oral administration of composition of the present invention has no apparent toxic or side-effects.

This experiment shows that the pharmaceutical composition of the present invention has high safety.

### II. Clinical Statistical Analysis

Products fabricated with composition of the present invention in Example 1 are taken as test products.

Take Gambir and Cassia, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.; dry the concentrated solution with high-speed centrifuging and spraying drier and make 80-120 meshes of dry extract powder; according to extraction ratio of Gambir and Cassia, mix the dry extract powder of the two evenly at the proportion of 15:5, add appropriate auxiliaries and make them into ordinary troches. Test product of composition of Gambir and Cassia has been made.

Mix the dry extract powder evenly at the proportion of 15 doses of Gambir, add appropriate auxiliaries and make them into ordinary troches. Test product of Gambir has been made.

Compare effects of taking composition of the present invention, composition of Gambir and Cassia and just Gambir. Dosage is 2 troches, 0.25 g in each troche (equals to 12 g of crude drug), before going to bed; analyze statistics of effects on 200 sleep apnea patients.

Symptoms of Sleep Apnea are Mainly:

Daytime: clinical symptoms are sleepiness, fatigue, hypomnesis, dyspnea chest discomfort, irritability, headache in the morning, dizziness and thirsty and throat ache. Standard symptoms shall be diagnosed by the pause times and duration of sleep apnea.

Nighttime: clinical symptoms are snoring, frequent pause of breathing, abnormal sleep actions; insomnia, dreaminess, labored breathing, polyuria and enuresis.

Degrees are mild, moderate and severe.

Mild patients suffer 30-50 times of apnea/seven hours, pause duration 10-30 seconds;

Moderate patients suffer 50-100 times of apnea/seven hours, pause duration 30-60 seconds;

Severe patients suffer 100-200 times of apnea/seven hours, pause duration 60+seconds;

Among the 240 patients, 180 are male and 60 are female, aging from 36-82. 120 of them are mild patients, 90 are moderate and 30 are severe. In the experiment, there are 3 groups: composition group of the present invention, group of Gambir+Cassia and group of Gambir. Each group has 80 patients, 60 of which are male, 20 are female, 40 are mild patients, 30 are moderate and 10 are severe.

Treatment: take the medicines with warm water every night before going to bed. 7 days are a therapy.

Evaluation standard for efficacy: effective: snoring sound lowered, times of awakening by labored breathing reduced, fatigue and restlessness during sleep disappeared, energetic during daytime, dizziness and tiredness disappeared.

Obvious efficacy: snoring sound lowered, thirstiness disappeared, never awakened by labored breathing, no sleepiness during daytime.

Cured: sleep well, snoring disappeared, complications caused by snoring disappeared, headache, dizziness, fatigue and sleepiness totally disappeared, energetic during daytime, clear thinking, memory enhanced. Totally recovered.

Effects Comparing:

| | | 1 therapy | | | | 2 therapies | | | |
|---|---|---|---|---|---|---|---|---|---|
| | | | Obvious | | Cured | | Obvious | | Cured |
| Degree | Medicine | Effective | efficacy | Cured | ratio | Effective | efficacy | Cured | ratio |
| Mild | Composition | 2 | 6 | 32 | 80% | 0 | 4 | 36 | 90% |
| patients | of the present | | | | | | | | |
| | invention | | | | | | | | |
| | Gambir + | 9 | 5 | 26 | 65% | 3 | 6 | 31 | 77.5% |
| | Cassia | | | | | | | | |
| | Gambir | 15 | 4 | 21 | 52.5% | 7 | 8 | 25 | 62.5% |
| Moderate | Composition | 3 | 9 | 18 | 60% | 1 | 4 | 25 | 83.3% |
| patients | of the present | | | | | | | | |
| | invention | | | | | | | | |
| | Gambir + | 12 | 7 | 11 | 36.7% | 5 | 6 | 19 | 63.3% |
| | Cassia | | | | | | | | |
| | Gambir | 15 | 6 | 9 | 30% | 7 | 7 | 16 | 53.3% |
| Severe | Composition | 2 | 5 | 3 | 30% | 1 | 3 | 6 | 60% |
| patients | of the present | | | | | | | | |
| | invention | | | | | | | | |
| | Gambir + | 5 | 4 | 1 | 10% | 2 | 5 | 3 | 30% |
| | Cassia | | | | | | | | |
| | Gambir | 6 | 4 | 0 | 0 | 4 | 4 | 2 | 20% |

No side-effects have been noticed during the therapy.

Compared with the group of Gambir+Cassia and Gambir, composition of the present invention has better efficacy. It takes effect faster, has no side-effects and is remarkably effective for snoring treatment.

### Experiment 2 Safety and Efficacy Evaluation of Composition of Gambir and Cassia

### I. Safety Evaluation

Products fabricated with composition of the present invention in Example 1 and 2 are used in the following experiment.

### 1. Acute Toxicity Test

Give intragastric administration of 50 g test product (crude drug)/kg to lab mouse at one time. The mouse didn't die during the experiment period. Thus it can be concluded that maximum tolerance dose to mouse at one time is 50 g (crude drug)/kg which equals 125 times of clinical daily dosage to human (2 capsules, which contains 12 g of crude drug, one time for man of 60 kg. Daily dosage for man is 24 g which is 0.4 g/kg).

### 2. Long Term Toxicity Test

Give lab mouse intragastric administration of dosage of 20 g/kg test product for 14 consecutive weeks. Compared with the control group that are given with distilled water, no clinical symptoms, weight gaining or changes in indicators in hematology and blood biochemical examination, toxicity in organs, system and pathological tissue have been noticed. Four weeks after drug withdrawal, no chronic toxicity has been noticed in the same examination items in the tested animal, which shows that consecutive oral administration of composition of the present invention has no apparent toxic or side-effects.

This experiment shows that the pharmaceutical composition of the present invention in Example 1 and 2 has high safety.

### II. Clinical Statistical Analysis

Products fabricated with composition of the present invention in Example 2 and 3 are taken as test products.

Take Gambir and Cassia, decoct respectively with water for three times. For the first time, add water amounting 6 times of the drugs and soak them for 1 hour, then decoct for 2 hours; for the second time, add water amounting 5 times of the drugs and decoct for 1 hours; for the third time, add water amounting 4 times of the drug, decoct for 1 hour, filter and put the filtrate together, heat and condense it to density ratio of 1.10-1.12 at the temperature of 60-65° C.; dry the concentrated solution with high-speed centrifuging and spraying drier and make 80-120 meshes of dry extract powder; according to extraction ratio of Gambir and Cassia, mix the dry extract powder of the two evenly at the proportion of 15:5, add appropriate auxiliaries and make them into ordinary troches. Test product of composition of Gambir and Cassia has been made.

Mix the dry extract powder evenly at the proportion of 15 doses of Gambir, add appropriate auxiliaries and make them into ordinary troches. Test product of Gambir has been made.

Compare effects of taking composition of the present invention, composition of Gambir and Cassia and drug of just Gambir. Dosage is 2 troches, 0.25 g in each troche (equals to 12 g of crude drug), before going to bed; analyze statistics of effects on 320 sleep apnea patients.

### Symptoms of Sleep Apnea are Mainly:

Daytime: clinical symptoms are sleepiness, fatigue, hypomnesis, dyspnea chest discomfort, irritability, headache in the morning, dizziness and thirsty and throat ache. Standard symptoms shall be diagnosed by the pause times and duration of sleep apnea.

Nighttime: clinical symptoms are snoring, frequent pause of breathing, abnormal sleep actions; insomnia, dreaminess, labored breathing, polyuria and enuresis.

Degrees are mild, moderate and severe.

Mild patients suffer 30-50 times of apnea/seven hours, pause duration 10-30 seconds;

Moderate patients suffer 50-100 times of apnea/seven hours, pause duration 30-60 seconds;

Severe patients suffer 100-200 times of apnea/seven hours, pause duration 60+seconds;

Among the 320 patients, 240 are male and 80 are female, aging from 36-82. 160 of them are mild patients, 1200 are moderate and 40 are severe. In the experiment, there are 4 groups: group given composition of the present invention in example 2, group given composition of the present invention in example 3, group of Gambir+Cassia and group of Gambir. Each group has 80 patients, 60 of which are male, 20 are female, 40 are mild patients, 30 are moderate and 10 are severe.

Treatment: take the medicines with warm water every night before going to bed. 7 days are a therapy.

Evaluation Standard for Efficacy:

Effective: snoring sound lowered, times of awakening by labored breathing reduced, fatigue and restlessness during sleep disappeared, energetic during daytime, dizziness and tiredness disappeared.

Obvious efficacy: snoring sound lowered, thirstiness disappeared, never awakened by labored breathing, no sleepiness during daytime.

Cured: sleep well, snoring disappeared, complications caused by snoring disappeared, headache, dizziness, fatigue and sleepiness totally disappeared, energetic during daytime, clear thinking, memory enhanced. Totally recovered.

Effects Comparing:

| | | 1 therapy | | | | 2 therapies | | | |
|---|---|---|---|---|---|---|---|---|---|
| | | | Obvious | | Cured | | Obvious | | Cured |
| Degree | Medicine | Effective | efficacy | Cured | ratio | Effective | efficacy | Cured | ratio |
| Mild | Composition | 3 | 5 | 32 | 80% | 1 | 2 | 37 | 92.5% |
| patients | fabricate in | | | | | | | | |
| | example 2 | | | | | | | | |
| | Composition | 2 | 5 | 33 | 82.5% | 0 | 3 | 37 | 92.5% |
| | fabricate in | | | | | | | | |
| | example 3 | | | | | | | | |
| | Gambir + | 9 | 5 | 26 | 65% | 3 | 6 | 31 | 77.5% |
| | Cassia | | | | | | | | |
| | Gambir | 15 | 4 | 21 | 52.5% | 7 | 8 | 25 | 62.5% |
| Moderate | Composition | 6 | 6 | 18 | 60% | 2 | 3 | 25 | 83.3% |
| patients | of the present | | | | | | | | |
| | invention | | | | | | | | |
| | Composition | 4 | 7 | 19 | 63.3% | 1 | 3 | 26 | 86.6% |
| | fabricate in | | | | | | | | |
| | example 3 | | | | | | | | |
| | Gambir + | 12 | 7 | 11 | 36.7% | 5 | 6 | 19 | 63.3% |
| | Cassia | | | | | | | | |
| | Gambir | 15 | 6 | 9 | 30% | 7 | 7 | 16 | 53.3% |
| Severe | Composition | 3 | 4 | 3 | 30% | 2 | 2 | 6 | 60% |
| patients | of the present | | | | | | | | |
| | invention | | | | | | | | |
| | Composition | 2 | 4 | 4 | 40% | 1 | 3 | 6 | 60% |
| | fabricate in | | | | | | | | |
| | example 3 | | | | | | | | |
| | Gambir + | 5 | 4 | 1 | 10% | 2 | 5 | 3 | 30% |
| | Cassia | | | | | | | | |
| | Gambir | 6 | 4 | 0 | 0 | 4 | 4 | 2 | 20% |

No side-effects have been noticed during the therapy.

Compared with the group of Gambir+Cassia and Gambir, compositions of the present invention in example 2 and 3 have better efficacy. They take effect faster, have no side-effects and are remarkably effective for snoring treatment.

### Experiment 3 Safety and Efficacy Evaluation of Composition of Gambir, Cassia Trichosanthes, Phragmites and Turmeric

### I. Safety Evaluation

Products fabricated with composition of the present invention in Example 6 are used in the following experiment.

### 1. Acute Toxicity Test

Give intragastric administration of 50 g test product (crude drug)/kg to lab mouse at one time. The mouse didn't die during the experiment period. Thus it can be concluded that maximum tolerance dose to mouse at one time is 50 g (crude drug)/kg which equals 125 times of clinical daily dosage to human (2 capsules, which contains 12 g of crude drug, one time for man of 60 kg. Daily dosage for man is 24 g which is 0.4 g/kg).

### 2. Long Term Toxicity Test

Give lab mouse intragastric administration of dosage of 20 g/kg for 14 consecutive weeks. Compared with the control group that are given with distilled water, no clinical symptoms, weight gaining or changes in indicators in hematology and blood biochemical examination, toxicity in organs, system and pathological tissue have been noticed. Four weeks after drug withdrawal, no chronic toxicity has been noticed in the same examination items in the tested animal, which shows that consecutive oral administration of composition of the present invention has no apparent toxic or side-effects.

### II. Clinical Statistical Analysis

Take products fabricated with composition of the present invention in Example 4. Dosage is 2 troches, 0.25 g in each troche (equals to 12 g of crude drug), before going to bed; analyze statistics of effects on 120 sleep apnea patients.

Symptoms of Sleep Apnea are Mainly:

Daytime: clinical symptoms are sleepiness, fatigue, hypomnesis, dyspnea chest discomfort, irritability, headache in the morning, dizziness and thirsty and throat ache. Standard symptoms shall be diagnosed by the pause times and duration of sleep apnea.

Nighttime: clinical symptoms are snoring, frequent pause of breathing, abnormal sleep actions; insomnia, dreaminess, labored breathing, polyuria and enuresis.

Degrees are mild, moderate and severe.

Mild patients suffer 30-50 times of apnea/seven hours, pause duration 10-30 seconds;

Moderate patients suffer 50-100 times of apnea/seven hours, pause duration 30-60 seconds;

Severe patients suffer 100-200 times of apnea/seven hours, pause duration 60+seconds;

Among the 120 patients, 80 are male and 40 are female, aging from 36-82. 60 of them are mild patients, 40 are moderate and 20 are severe.

Treatment: take the medicines with warm water every night before going to bed. 7 days are a therapy.

Evaluation standard for efficacy: effective: snoring sound lowered, times of awakening by labored breathing reduced, fatigue and restlessness during sleep disappeared, energetic during daytime, dizziness and tiredness disappeared.

Obvious efficacy: snoring sound lowered, thirstiness disappeared, never awakened by labored breathing, no sleepiness during daytime.

Cured: sleep well, snoring disappeared, complications caused by snoring disappeared, headache, dizziness, fatigue and sleepiness totally disappeared, energetic during daytime, clear thinking, memory enhanced. Totally recovered.

Treatment Effects

| | | 1 therapy | | | | 2 therapies | | | |
|---|---|---|---|---|---|---|---|---|---|
| | | | Obvious | | Cured | | Obvious | | Cured |
| Degree | Medicine | Effective | efficacy | Cured | ratio | Effective | efficacy | Cured | ratio |
| Mild | Composition | 3 | 4 | 53 | 88.3% | 0 | 3 | 57 | 95% |
| patients | of the present | | | | | | | | |
| | invention | | | | | | | | |
| Moderate | Composition | 5 | 7 | 28 | 70% | 1 | 3 | 36 | 87.5% |
| patients | of the present | | | | | | | | |
| | invention | | | | | | | | |
| Severe | Composition | 4 | 7 | 9 | 45% | 1 | 5 | 14 | 70% |
| patients | of the present | | | | | | | | |
| | invention | | | | | | | | |

No side-effects have been noticed during the therapy.

Treatment effects show that composition of the present invention has good efficacy. It takes effect fast, has no side-effects, has no side-effects and is remarkably effective for snoring treatment. Cured ratios of mild, moderate and severe patients are respectively 88.3%, 70%, 45% after 1 therapy of the composition, and 95%, 87.5%, 70% after 1 therapies, while cured ratios mild, moderate and severe patients are respectively 80%, 60%, 30% after 1 therapy of composition of Gambir+Cassia and 90%, 83.3%, 60% after 2 therapies. Therefore, treatment effects of composition of the five drugs are better than that of composition of just two.

### Experiment 4 Safety and Efficacy Evaluation of Composition of Gambir, Cassia, Trichosanthes, Phragmites and Turmeric

### I. Safety Evaluation

Products fabricated with composition of the present invention in Example 4 and 5 are used in the following experiment.

### 1. Acute Toxicity Test

Give intragastric administration of 50 g test product (crude drug)/kg to lab mouse at one time. The mouse didn't die during the experiment period. Thus it can be concluded that maximum tolerance dose to mouse at one time is 50 g (crude drug)/kg which equals 125 times of clinical daily dosage to human (2 capsules, which contains 12 g of crude drug, one time for man of 60 kg. Daily dosage for man is 24 g which is 0.4 g/kg).

### 2. Long Term Toxicity Test

Give lab mouse intragastric administration of dosage of 20 g/kg test product for 14 consecutive weeks. Compared with the control group that are given with distilled water, no clinical symptoms, weight gaining or changes in indicators in hematology and blood biochemical examination, toxicity in organs, system and pathological tissue have been noticed. Four weeks after drug withdrawal, no chronic toxicity has been noticed in the same examination items in the tested animal, which shows that consecutive oral administration of composition of the present invention has no apparent toxic or side-effects.

This experiment shows that the pharmaceutical composition of the present invention in Example 4 and 5 has high safety.

### II. Clinical Statistical Analysis

Take composition of the present invention (fabricated in Example 4 and 5) as test products.

Dosage is 2 troches, 0.25 g in each troche (equals to 12 g of crude drug), before going to bed; analyze statistics of effects on 240 sleep apnea patients.

Symptoms of Sleep Apnea are Mainly:

Daytime: clinical symptoms are sleepiness, fatigue, hypomnesis, dyspnea chest discomfort, irritability, headache in the morning, dizziness and thirsty and throat ache. Standard symptoms shall be diagnosed by the pause times and duration of sleep apnea.

Nighttime: clinical symptoms are snoring, frequent pause of breathing, abnormal sleep actions; insomnia, dreaminess, labored breathing, polyuria and enuresis.

Degrees are mild, moderate and severe.

Mild patients suffer 30-50 times of apnea/seven hours, pause duration 10-30 seconds;

Moderate patients suffer 50-100 times of apnea/seven hours, pause duration 30-60 seconds; Severe patients suffer 100-200 times of apnea/seven hours, pause duration 60+seconds;

Among the 240 patients, 160 are male and 80 are female, aging from 36-82. 120 of them are mild patients, 80 are moderate and 40 are severe. In the experiment, there are 2 groups: group given composition of the present invention in example 5 and group given composition of the present invention in example 6. Each group has 120 patients, 80 of which are male, 40 are female, 60 are mild patients, 40 are moderate and 20 are severe.

Treatment: take the medicines with warm water every night before going to bed. 7 days are a therapy.

Evaluation Standard for Efficacy:

Effective: snoring sound lowered, times of awakening by labored breathing reduced, fatigue and restlessness during sleep disappeared, energetic during daytime, dizziness and tiredness disappeared.

Obvious efficacy: snoring sound lowered, thirstiness disappeared, never awakened by labored breathing, no sleepiness during daytime.

Cured: sleep well, snoring disappeared, complications caused by snoring disappeared, headache, dizziness, fatigue and sleepiness totally disappeared, energetic during daytime, clear thinking, memory enhanced. Totally recovered.

Effects Comparing:

| | | 1 therapy | | | | 2 therapies | | | |
|---|---|---|---|---|---|---|---|---|---|
| | | | Obvious | | Cured | | Obvious | | Cured |
| Degree | Medicine | Effective | efficacy | Cured | ratio | Effective | efficacy | Cured | ratio |
| Mild | Composition | 2 | 4 | 54 | 91.6% | 1 | 2 | 57 | 95% |
| patients | fabricated in | | | | | | | | |
| | example 5 | | | | | | | | |
| | Composition | 1 | 5 | 54 | 91.6% | 0 | 2 | 58 | 96.7% |
| | fabricated in | | | | | | | | |
| | example 6 | | | | | | | | |
| Moderate | Composition | 5 | 7 | 28 | 70% | 2 | 2 | 36 | 90% |
| patients | fabricated in | | | | | | | | |
| | example 5 | | | | | | | | |
| | Composition | 3 | 8 | 29 | 72.5% | 2 | 2 | 36 | 90% |
| | fabricated in | | | | | | | | |
| | example 6 | | | | | | | | |
| Severe | Composition | 5 | 5 | 10 | 50% | 2 | 4 | 14 | 70% |
| patients | fabricated in | | | | | | | | |
| | example 5 | | | | | | | | |
| | Composition | 3 | 7 | 10 | 50% | 1 | 5 | 14 | 70% |
| | fabricated in | | | | | | | | |
| | example 6 | | | | | | | | |

No side-effects have been noticed during the therapy.

Treatment effects show that compositions of the present invention in example 5 and 6 have good efficacy. They take effect fast, have no side-effects, and are remarkably effective for snoring treatment. Cured ratios of mild, moderate and severe patients are respectively 91.6%, 70%, 50% after 1 therapy of composition in example 5, and 95%, 90%, 70% after 2 therapies; 91.6%, 72.5%, 50% after 1 therapy of composition in example 6 and 96.7%, 90%, 70% after 2 therapies; 80%, 60% and 30% after 1 therapy of composition of Gambir and Cassia and 90%, 83.3% and 60% after 2 therapies. Therefore, treatment effects of composition of the five drugs in example 5 and 6 are better than that of composition of just two of Gambir and Cassia.

What is claimed is:

1. A pharmaceutical composition for snoring treatment features that its crude drugs contain components proportioned by weight as follows: 15-25 doses of Gambir and 5-15 doses of Cassia.

2. The pharmaceutical composition mentioned above in claim 1 features that its crude drugs contain components proportioned by weight as follows: 15 doses of Gambir and 5 doses of Cassia; 20 doses of Gambir and 10 doses of Cassia; 25 doses of Gambir and 15 doses of Cassia.

3. The pharmaceutical composition mentioned above in claim 1 features that its crude drugs contain components proportioned by weight as follows: 12-18 doses of Trichosanthes, 12-20 doses of Phragmites and 6-12 doses of Turmeric.

4. The pharmaceutical composition mentioned above in claim 3 features that its crude drugs contain components proportioned by weight as follows:
15 doses of Gambir, 5 doses of Cassia, 12 doses of Trichosanthes, 12 doses of Phragmites and 6 doses of Turmeric;
20 doses of Gambir, 10 doses of Cassia, 15 doses of Trichosanthes, 17 doses of Phragmites and 9 doses of Turmeric;
25 doses of Gambir, 15 doses of Cassia, 18 doses of Trichosanthes, 20 doses of Phragmites and 12 doses of Turmeric;

5. The pharmaceutical composition mentioned above in claim 1 features that it is a dosage form of drug fabricated by combing the active ingredients such as power, aqueous extract and alcohol extract of the crude drugs with common pharmaceutical auxiliaries or auxiliary materials.

6. The dosage form of drug mentioned above in claim 5 features that it is a gastrointestinal absorption form of drug.

7. Fabrication of pharmaceutical compositions mentioned in claims 1-4 features that it includes the following steps:
(1) The crude drugs are taken in certain proportions;
(2) Take the crude drugs and mix them up for aqueous extraction, or extract the crude drugs respectively. Then take the aqueous extractives out for use in next step.
(3) Add common pharmaceutical auxiliaries or auxiliary materials into the aqueous extractives and the composition is made.

8. Fabrication mentioned above in claim 7 features that the extraction is made by decoction or ultrasound.

9. Any of the pharmaceutical compositions mentioned in claims 1-4 are useful for fabricating drugs for snoring treatment.

10. The pharmaceutical composition mentioned above in claim 2 features that it is a dosage form of drug fabricated by combing the active ingredients such as power, aqueous extract and alcohol extract of the crude drugs with common pharmaceutical auxiliaries or auxiliary materials.

11. The pharmaceutical composition mentioned above in claim 3 features that it is a dosage form of drug fabricated by combing the active ingredients such as power, aqueous extract and alcohol extract of the crude drugs with common pharmaceutical auxiliaries or auxiliary materials.

12. The pharmaceutical composition mentioned above in claim 4 features that it is a dosage form of drug fabricated by combing the active ingredients such as power, aqueous extract and alcohol extract of the crude drugs with common pharmaceutical auxiliaries or auxiliary materials.

13. The dosage form of drug mentioned above in claim 10 features that it is a gastrointestinal absorption form of drug.

14. The dosage form of drug mentioned above in claim 11 features that it is a gastrointestinal absorption form of drug.

15. The dosage form of drug mentioned above in claim 12 features that it is a gastrointestinal absorption form of drug.
